(19) United States
(12) Patent Application Publication
(10) Pub. No.: US 2026/0187876 A1
(43) Pub. Date: Jul. 2, 2026

(54) IMAGE GENERATION APPARATUS, TRAINING APPARATUS, IMAGE GENERATION METHOD, AND STORAGE MEDIUM

(71) Applicant: NEC CORPORATION, Tokyo (JP)

(72) Inventors: Shigeaki NAMIKI, Tokyo (JP); Yuji IWADATE, Tokyo (JP)

(73) Assignee: NEC CORPORATION, Tokyo (JP)

(21) Appl. No.: 19/131,575

(22) PCT Filed: Nov. 30, 2022
(86) PCT No.: PCT/JP2022/044109

§ 371 Date: May 21, 2025

(51) Int. Cl. G06T 11/60; G06V 10/774

(52) U.S. Cl. CPC G06T 11/60; G06V 10/774; G06T 2210/62

(57) ABSTRACT

In order to attain an example object of reducing influence of a feature of a processing process in training data on accuracy of machine learning, an image generation apparatus (1) includes a setting section (11) for randomly setting a processing parameter; a processing section (12) for processing one or both of a first image and a second image in accordance with the processing parameter; and a generation section (13) for pasting the first image to the second image so as to generate a third image that serves as training data for machine learning.

### TECHNICAL FIELD

The present invention relates to a technique to generate an image which is to be used as training data.

### BACKGROUND ART

A technique to generate an image which is to be used as training data is known. Such a technique is used, for example, for the purpose of increasing the number of pieces of training data so as to improve accuracy of machine learning. For example, Non-Patent Literature 1 discloses a technique to generate an image which is to be used as training data by pasting a foreground which is a region where a margin is added to a lesion region in a medical image to another medical image which serves as a background. In this technique, pixel values of the margin in the foreground are smoothly changed, and then the foreground is pasted to the medical image serving as a background.

### CITATION LIST

### Non-Patent Literature

### Non-patent Literature 1

Pingping Dai et. al., “Soft-CP: A Credible and Effective Data Augmentation for Semantic Segmentation of Medical Lesions”, 2203.10507.pdf (arxiv.org)

### SUMMARY OF INVENTION

### Technical Problem

In the technique disclosed in Non-Patent Literature 1, a margin is added along a shape of a lesion region. Therefore, in a case where machine learning is carried out while using, as training data, a medical image generated by the technique, a feature of discontinuity in a boundary between (i) a margin along a shape of a lesion region and (ii) a background may be learned. For example, an image recognition model trained while using, as training data, a medical image generated by the technique may recognize, as a lesion region, a region which is similar to a shape of a lesion region and has discontinuity in a boundary of the region even in a case where the region does not include a lesion. Thus, the training data generated by the technique disclosed in Non-Patent Literature 1 has a problem that a feature of a processing process remaining in the training data affects accuracy of machine learning.

An example aspect of the present invention is accomplished in view of the above problem, and an example object thereof is to provide a technique which reduces influence of a feature of a processing process remaining in training data on accuracy of machine learning.

### Solution to Problem

An image generation apparatus in accordance with an example aspect of the present invention includes: a setting means for randomly setting a processing parameter; a processing means for processing one or both of a first image and a second image in accordance with the processing parameter; and a generation means for pasting the first image to the second image so as to generate a third image that serves as training data for machine learning.

An image generation method in accordance with an example aspect of the present invention includes: randomly setting, by at least one processor, a processing parameter; processing, by the at least one processor, one or both of a first image and a second image in accordance with the processing parameter; and pasting, by the at least one processor, the first image to the second image so as to generate a third image that serves as training data for machine learning.

A program in accordance with an example aspect of the present invention causes at least one processor to function as: a setting means for randomly setting a processing parameter; a processing means for processing one or both of a first image and a second image in accordance with the processing parameter; and a generation means for pasting the first image to the second image so as to generate a third image that serves as training data for machine learning.

### Advantageous Effects of Invention

According to an example aspect of the present invention, it is possible to reduce influence of a feature of a processing process in training data on accuracy of machine learning.

### BRIEF DESCRIPTION OF DRAWINGS

FIG. 1 is a block diagram illustrating a configuration of an image generation apparatus in accordance with a first example embodiment.

FIG. 2 is a flowchart illustrating a flow of an image generation method in accordance with the first example embodiment.

FIG. 3 is a block diagram illustrating a configuration of an information processing system in accordance with a second example embodiment.

FIG. 4 is a flowchart for describing a flow of an image generation method in accordance with the second example embodiment.

FIG. 5 is a schematic diagram illustrating an example of a first region, a second region, and an augmented medical image.

FIG. 6 is a schematic diagram illustrating a setting example of transmittancy.

FIG. 7 is a schematic diagram illustrating an example of an augmented medical image to which a dummy region is pasted.

FIG. 8 is a schematic diagram illustrating a setting example of transmittancy.

FIG. 9 is a block diagram illustrating a hardware configuration example of the image generation apparatus in accordance with each of the example embodiments and the training apparatus in accordance with the second example embodiment.

### First Example Embodiment

The following description will discuss a first example embodiment of the present invention in detail, with reference to the drawings. The present example embodiment is a basic form of example embodiments described later.

### <Configuration of Image Generation Apparatus 1>

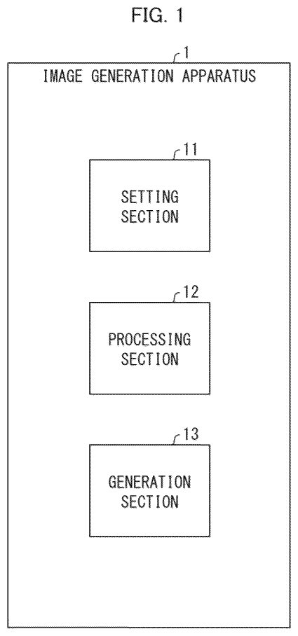

The following description will discuss a configuration of an image generation apparatus 1 in accordance with the present example embodiment, with reference to FIG. 1. FIG. 1 is a block diagram illustrating the configuration of the image generation apparatus 1. As illustrated in FIG. 1, the image generation apparatus 1 includes a setting section 11, a processing section 12, and a generation section 13. The setting section 11 is an example configuration for implementing the setting means recited in claims. The processing section 12 is an example configuration for implementing the processing means recited in claims. The generation section 13 is an example configuration for implementing the generation means recited in claims.

The setting section 11 randomly sets a processing parameter. The processing section 12 processes one or both of a first image and a second image in accordance with the processing parameter. The generation section 13 pastes the first image to the second image so as to generate a third image that serves as training data for machine learning.

### <Example of Implementation by Program>

In a case where the image generation apparatus 1 is implemented by a computer, the following program in accordance with the present example embodiment is stored in a memory of the computer. The program causes the computer to function as: the setting section 11 for randomly setting a processing parameter; the processing section 12 for processing one or both of a first image and a second image in accordance with the processing parameter; and the generation section 13 for pasting the first image to the second image so as to generate a third image that serves as training data for machine learning.

### <Flow of Image Generation Method S1>

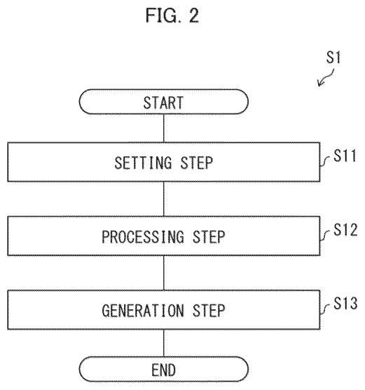

The image generation apparatus 1 configured as described above carries out an image generation method S1 in accordance with the present example embodiment. The following description will discuss a flow of the image generation method S1, with reference to FIG. 2. FIG. 2 is a flowchart illustrating the flow of the image generation method S1. As illustrated in FIG. 2, the image generation method S1 includes a setting step S11, a processing step S12, and a generation step S13.

In the setting step S11, the setting section 11 randomly sets a processing parameter. In the processing step S12, the processing section 12 processes one or both of a first image and a second image in accordance with the processing parameter. In the generation step S13, the generation section 13 pastes the first image to the second image so as to generate a third image that serves as training data for machine learning.

### <Example Advantage of Present Example Embodiment>

As described above, the image generation apparatus 1 and the image generation method S1 in accordance with the present example embodiment employs the configuration of: randomly setting a processing parameter; processing one or both of a first image and a second image in accordance with the set processing parameter; and pasting the first image to the second image so as to generate a third image that serves as training data for machine learning. Therefore, according to the present example embodiment, it is possible to bring about an example advantage of reducing influence of a feature of a processing process in training data on accuracy

### Second Example Embodiment

The following description will discuss a second example embodiment of the present invention in detail, with reference to the drawings. An information processing system 100 in accordance with the present example embodiment is a system that carries out machine learning of an image recognition model ML with respect to medical images. In a case of carrying out machine learning of the image recognition model ML, a medical image including a region indicating a lesion (hereinafter referred to also as a lesion region) is needed as training data. However, there is a case where it is difficult to collect a large number of medical images each including a lesion region. For example, it is difficult to sufficiently collect medical images of a rare-case lesion. Under the circumstances, the information processing system 100 generates an augmented medical image by augmenting a medical image including a lesion region, and carries out machine learning while using, as training data, the original medical image and the generated augmented medical image. In the descriptions of the present example embodiment below, the same reference numerals are given to constituent elements which have functions identical with those described in the first example embodiment, and descriptions as to such constituent elements are omitted as appropriate.

In training data in the present example embodiment, a label is associated with each pixel in a medical image. The label is assumed to have a value 1 which indicates a lesion part, a value 0 which indicates a non-lesion part, and a value of 0 or more and 1 or less which indicates a degree of being a lesion. The image recognition model ML to be trained using the training data is a model which carries out a segmentation task of recognizing a lesion region for each pixel.

### <Configuration of Information Processing System 100>

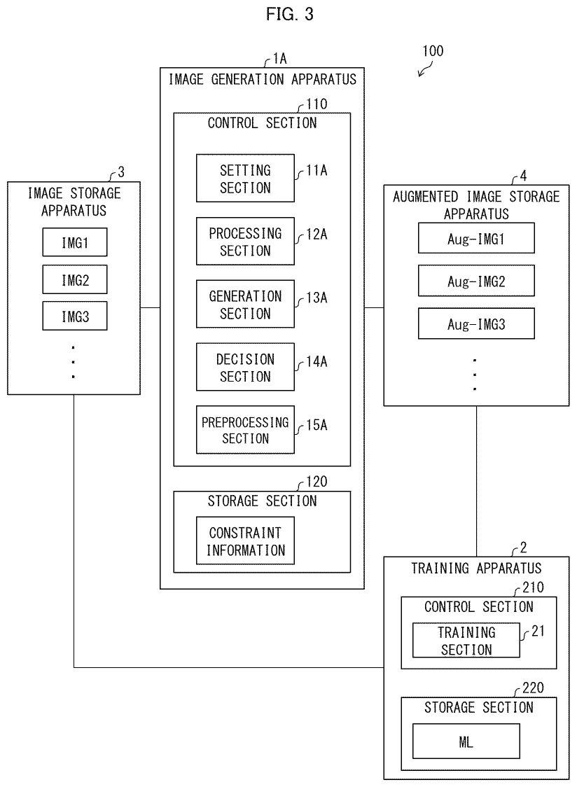

The following description will discuss a configuration of the information processing system 100 in accordance with the present example embodiment, with reference to FIG. 3. FIG. 3 is a block diagram illustrating the configuration of the information processing system 100. As illustrated in FIG. 3, the information processing system 100 includes an image generation apparatus 1A, a training apparatus 2, an image storage apparatus 3, and an augmented image storage apparatus 4. The image generation apparatus 1A is connected to the image storage apparatus 3 and the augmented image storage apparatus 4 so as to be capable of reading and writing information therefrom and thereto. The training apparatus 2 is connected to the image storage apparatus 3 and the augmented image storage apparatus 4 so as to be capable of reading information therefrom. Note that these apparatuses may be communicably connected to each other via a network.

The image storage apparatus 3 stores a plurality of medical images IMG1, IMG2, IMG3, and so forth each obtained by imaging a predetermined part of a human body. In a case where it is not necessary to particularly distinguish between the medical images IMG1, IMG2, IMG3, and so forth, the images are each simply referred to as a medical image. Examples of types of medical images include, but not limited to, an endoscopic image, an ultrasonic image, a computed tomography (CT) image, a magnetic resonance imaging (MRI) image, and the like. Examples of a predetermined part of a human body include, but not limited to, a bowel, a stomach, a gullet, a lung, a head, and the like. The plurality of medical images are medical images of the same predetermined type (e.g., endoscopic images) obtained by imaging the same predetermined part (e.g., a bowel).

The plurality of medical images include a medical image which includes a lesion region and a medical image which does not include a lesion region. Each of the medical images is associated with labels. A label is associated with each pixel in a medical image. For example, each pixel included in a lesion region is associated with a label “1” indicating a lesion, and each pixel included in the other region is associated with a label “0” indicating a non-lesion part. Each of medical images including a lesion region can be an example of the first image recited in claims. Moreover, each of medical images can be an example of the second image, regardless of whether or not the image includes a lesion region. Hereinafter, the first image is referred to as a foreground image, and the second image is referred to as a background image.

The augmented image storage apparatus 4 stores augmented medical images Aug-IMG1, Aug-IMG2, Aug-IMG3, and so forth which have been generated by the image generation apparatus 1A. In a case where it is not necessary to particularly distinguish between the augmented medical images Aug-IMG1, Aug-IMG2, Aug-IMG3, and so forth, the images are each simply referred to as an augmented medical image. The augmented medical image is an example of the third image recited in claims.

The plurality of augmented medical images each include a lesion region. Each of the augmented medical images is associated with labels which have been decided by the image generation apparatus 1A. The label is decided for and associated with each pixel in an augmented medical image.

The image generation apparatus 1A includes a control section 110 and a storage section 120. The control section 110 comprehensively controls the sections of the image generation apparatus 1A. The storage section 120 stores a program for enabling the control section 110 to function, and various kinds of data used by the control section 110. For example, the storage section 120 stores constraint information. The constraint information is information indicating a constraint that pertains to a pasting position corresponding to a predetermined part which is included in a medical image as a subject.

The control section 110 includes a setting section 11A, a processing section 12A, a generation section 13A, a decision section 14A, and a preprocessing section 15A. The decision section 14A is an example configuration for implementing the decision means recited in claims.

The setting section 11A is configured as below, in addition to have a configuration similar to that of the setting section 11. That is, the setting section 11A randomly sets a processing parameter including at least one selected from the group consisting of (1) a parameter which designates a shape of one or both of the first region and the second region, (2) a parameter which defines a size of one or both of the first region and the second region, and (3) a parameter which defines image processing. The image processing is, for example, smoothing or transparentizing. The parameter which defines image processing can be a parameter which defines a type of algorithm (e.g., a type of smoothing filter or the like) with which image processing is carried out. The other parameter which defines image processing can be a parameter which defines a mode of image processing (e.g., a mode of alpha blending). Still another parameter which defines image processing can be a parameter which defines intensity of image processing (e.g., a kernel size of smoothing filter, transmittancy of alpha blending). Note, however, that the parameters which define image processing are not limited to the above described examples.

The first region is a region including a boundary of a lesion region which is associated with a label indicating a lesion in a foreground image. The second region is a region including a boundary of a region which, in a background image, overlaps with the lesion region in a case where the foreground image is pasted. Note that the label indicating a lesion is an example of the “predetermined label” recited in claims. The lesion region is an example of the “predetermined region which is associated with a predetermined label”.

The processing section 12A is configured as below, in addition to have a configuration similar to that of the processing section 12. That is, the processing section 12A applies image processing to one or both of the first region and the second region so as to process one or both of the foreground image and the background image. The processing section 12, as image processing to be applied to one or both of the first region and the second region, may apply one or both of the foregoing smoothing and transparentizing. In the present example embodiment, it is assumed that smoothing and transparentizing are both applied.

The generation section 13A is configured as below, in addition to have a configuration similar to that of the generation section 13. That is, the generation section 13A decides a label to be associated with the generated augmented medical image by referring to a label associated with the foreground image. For example, the generation section 13A decides a label to be associated with the generated augmented medical image by referring to a label associated with the background image and the processing parameter, in addition to the label associated with the foreground image.

The decision section 14A decides, by referring to the background image, a position at which the foreground image is pasted to the background image. The decision section 14A decides, by further referring to constraint information indicating a constraint which pertains to a pasting position corresponding to the predetermined part, a position at which the foreground image is pasted to the background image, in addition to referring to the background image. The position of the pasting is hereinafter referred to also as a “pasting position”.

The preprocessing section 15A applies image processing to one or both of the foreground image and the background image. The image processing carried out by the preprocessing section 15A is different from that carried out by the processing section 12A, and is carried out before the processing section 12A carries out the image processing. The image processing carried out by the preprocessing section 15A is referred to also as a preprocessing process.

The training apparatus 2 includes a control section 210 and a storage section 220. The control section 210 comprehensively, sections of the training apparatus 2. The storage section 220 stores a program for enabling the control section 210 to function, and various kinds of data used by the control section 210. The control section 210 includes a training section 21. The training section 21 trains the image recognition model ML using training data which has been generated by using the image generation apparatus 1A. The storage section 220 stores the image recognition model ML.

### <Flow of Image Generation Method S1A>

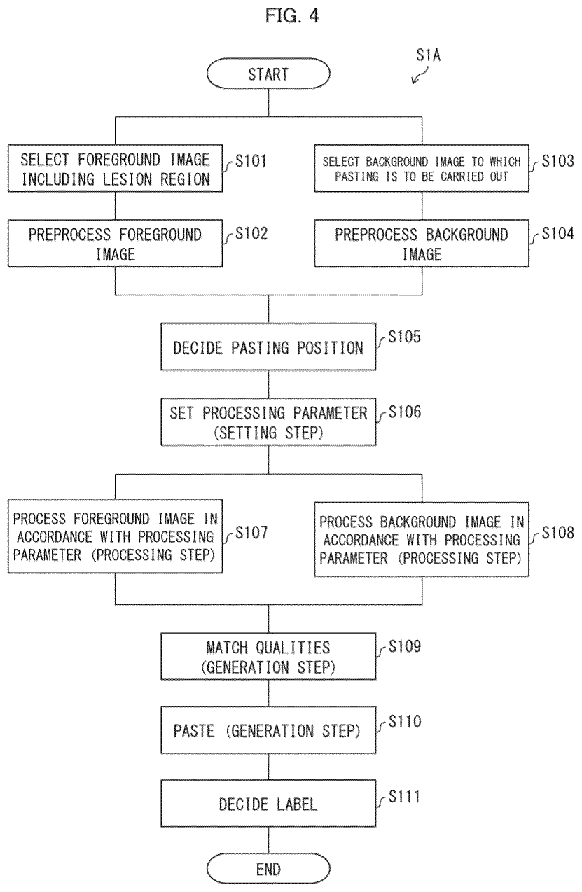

The following description will discuss a flow of an image generation method S1 that is carried out by the image generation apparatus 1A configured as described above, with reference to FIG. 4. FIG. 4 is a flowchart illustrating the flow of the image generation method S1. As illustrated in FIG. 4, the image generation method S1A includes steps S101 through S111.

In step S101, the control section 110 selects and reads a foreground image from among medical images stored in the image storage apparatus 3. Specifically, the control section 110 selects, as the foreground image, a medical image (in other words, a medical image including pixels associated with labels each indicating a lesion) which includes a lesion region.

In step S102, the preprocessing section 15A applies a preprocessing process to the foreground image. Examples of the preprocessing process include, but not limited to, geometric image processing, optical image processing, a process of adding noise, and the like. As the preprocessing process, it is possible to employ various known techniques which are used to generate, from an image serving as training data, the other training data. For example, the preprocessing section 15A changes a label, if necessary, in accordance with the preprocessing process. Specifically, for example, in a case where the preprocessing process is geometric image processing, the preprocessing section 15A, according to geometric transformation of a medical image, associates a label which is associated with a pixel before transformation with a pixel after transformation which corresponds to the pixel before transformation. Such a preprocessing process is carried out based on a parameter which has been determined in advance.

In step S103, the control section 110 selects and reads, from among medical images stored in the image storage apparatus 3, a background image to which pasting is to be carried out. The background image only needs to be an image different from the foreground image, and may be an image which includes a lesion region or may be an image which does not include a lesion region.

In step S104, the preprocessing section 15A applies a preprocessing process to the background image. The details of the preprocessing process are similar to step S102, and therefore detailed descriptions will not be repeated. Note that the processes of steps S101 and S102 and the processes of steps S103 and S104 may be carried out in parallel or may be carried out in the reverse order.

In step S105, the decision section 14A decides, by referring to the background image and constraint information according to the predetermined part, a pasting position of the foreground image in the background image. The decision section 14A may decide the pasting position by further referring to a lesion region in the foreground image, in addition to the background image and the constraint information. Note that the constraint information is, as described above, information indicating a constraint that pertains to a pasting position corresponding to a predetermined part which is included in a medical image as a subject. The constraint information may be stored in the storage section 120 or may be stored in an external apparatus.

### (Specific Example of Constraint Information)

The following description will discuss a specific example of constraint information. Here, it is assumed that the image storage apparatus 3 stores an endoscopic image of a bowel as a medical image. That is, the predetermined part is a bowel. In this case, the constraint information is information indicating a constraint that pertains to a pasting position corresponding to the endoscopic image of the bowel. For example, the endoscopic image of the bowel may include a region indicating an intestinal wall and a region indicating an intestinal tract. An image obtained by imaging the region indicating an intestinal tract often has lower luminance because the imaged region indicating an intestinal tract is deeper than the region indicating an intestinal wall. Such a region with lower luminance is less likely to include a lesion region. Therefore, such a region indicating an intestinal tract is not appropriate as a region to which a lesion region is to be pasted. An endoscopic image is a rectangle image including a circular region imaged by an endoscope. Therefore, the endoscopic image may include a region which is out of imaging range at at least one of the four corners. Such a region which is out of imaging range is also not appropriate as a region to which a lesion region is to be pasted. Thus, the constraint information includes information indicating a region (hereinafter referred to also as a pasting-impossible region) to which it is not appropriate to paste a lesion region. Information indicating a pasting-impossible region may be a mask image. For example, the mask image may be an image in which regions at four corners of the image which are out of imaging range of an endoscope are masked and a region near a center of the image which is more likely to indicate an intestinal tract is also masked.

An endoscopic image which already includes a lesion region can be used as a background image. In this case, the lesion region is not appropriate as a region to which another lesion region is to be pasted. In this case, information indicating a pasting-impossible region may be an image recognition model which recognizes a pasting-impossible region. Such an image recognition model can be, for example, a model which has been separately trained by machine learning.

The decision section 14, for example, identifies a pasting-impossible region in a background image by referring to the background image and constraint information. The decision section 14 decides, in the background image, a pasting position of a foreground image so that a lesion region in the foreground image is pasted in a region other than the pasting-impossible region in the background image. At this time, the decision section 14 refers to also a shape and a size of the lesion region in the foreground image. Note that, in a region other than the pasting-impossible region, the decision section 14 randomly decides a pasting position of a lesion region.

In step S106, the setting section 11A decides (1) a parameter which designates a shape of one or both of a first region in the foreground image and a second region in the background image, (2) a parameter which defines a size of one or both of the first region and the second region, and (3) a parameter which defines the image processing.

### (Example of Setting Parameters Defining Shape and Size)

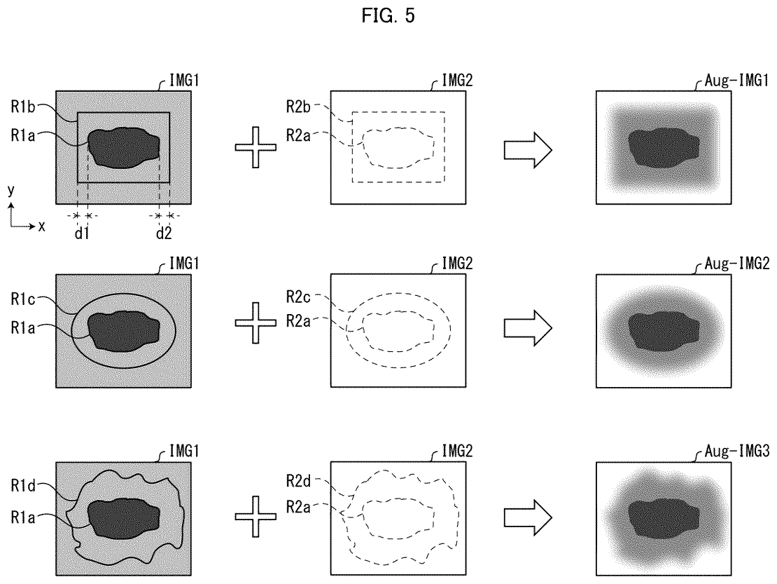

The following description will discuss an example of a first region and a second region, with reference to FIG. 5. FIG. 5 is a schematic diagram illustrating an example of a first region, a second region, and an augmented medical image. In the example illustrated in FIG. 5, an image IMG1 is a medical image selected as a foreground image. In the image IMG1, a region surrounded by a boundary R1a indicates a lesion region. That is, pixels inside the boundary R1a are respectively associated with labels indicating a lesion. The first region is set as a region including the boundary R1a of the lesion region in the image IMG1. In FIG. 5, the first region is an annular region which has the boundary R1a as an inner boundary and a boundary R1b (or R1c or R1d) as an outer boundary. Such an annular first region is referred to also as a margin.

In FIG. 5, an image IMG2 is a medical image selected as a background image. The second region is set as a region including a boundary R2a. The boundary R2a is a boundary of a region which, in the image IMG2, overlaps with the lesion region in a case where the image IMG1 is pasted at the pasting position. In FIG. 5, the second region is an annular region which has the boundary R2a as an inner boundary and a boundary R2b (or R2c or R2d) as an outer boundary. Such an annular second region is referred to also as a margin.

FIG. 5 indicates an example in which, as the second region, a region is set which is the same as a region that overlaps with the first region. In other words, boundaries R1b, R1c, and R1d in the image IMG1 overlap with respective boundaries R2b, R2c, and R2d in the image IMG2 in a case where the image IMG1 is pasted to the image IMG2 at the pasting position. The following description will discuss an example in which, as illustrated in FIG. 5, “the second region is the same as a region which overlaps with the first region”, and the first region and the second region are each referred to as a “margin”.

For example, the setting section 11A may randomly decide, as a shape of the margin, any of a plurality of types of shapes which have been determined in advance. Examples of such types of shapes which have been determined in advance include a rectangle such as that of the boundary R1b, an ellipse such as that of the boundary R1c, an arbitrary shape such as that of the boundary R1d, and the like. The arbitrary shape can be, for example, a shape indicated by the boundary R1d that is formed by connecting points that are apart, by a randomly set distance, from respective points on the boundary R1a of the lesion region to the outside. In this case, a distance from a certain point on the boundary R1a to the boundary R1d can differ from a distance from another point on the boundary R1a to the boundary R1d. Therefore, the boundary R1d set in such a manner can have an arbitrary shape that is surrounded by curves with recesses and protrusions as illustrated in FIG. 5. Other examples of the types of shapes which have been determined in advance include, but not limited to, various polygons, a shape similar to a lesion region, and the like.

For example, the setting section 11A may randomly decide, as a size of the margin, any numerical value from a numerical range which has been determined in advance. A parameter to be decided as a size and a numerical range thereof may be set in advance in accordance with a shape of the margin. Examples of the parameter to be decided as a size include a length and a width of a rectangle, a major axis and a minor axis of an ellipse, a distance from each point on the boundary R1a of the lesion region in the foregoing arbitrary shape.

For example, the setting section 11A may randomly decide a position of the margin as still another processing parameter. In a case where a position of the margin is randomly set, the margin does not necessarily need to include a lesion region in a substantially center thereof. For example, the following description will discuss a case where a shape of the margin is a rectangle where a direction from the left to the right in FIG. 5 is an x-axis positive direction, and a direction from the bottom to the top in FIG. 5 is a y-axis positive direction. In this case, (i) a distance d1 from a point at which an x-coordinate on the boundary R1a of the lesion region is minimum to a point at which an x-coordinate on the outer boundary R1b of the margin is minimum and (ii) a distance d2 from a point at which the x-coordinate on the boundary R1a is maximum to a point at which the x-coordinate on the boundary R1b is maximum are randomly decided. These distances do not necessarily need to be equal to each other. The same applies to the y-axis direction. Moreover, the same applies to a margin in another shape.

### (Example of Setting Parameter Defining Image Processing)

The following description will discuss a specific example of a parameter which defines image processing. Here, as described above, the image processing is smoothing or transparentizing. For example, the setting section 11A randomly sets, as a processing parameter, a type of smoothing filter for smoothing. Specifically, the setting section 11A may carry out setting by randomly selecting one of a plurality of types of smoothing filters (e.g., Gaussian filter, moving-average filter, median filter, and the like) which have been determined in advance. For example, the setting section 11A randomly sets a parameter which defines intensity of the set smoothing filter. Specifically, the setting section 11A may set a kernel size to be used in the smoothing filter by randomly selecting a numerical value in a numerical range which has been determined in advance.

For example, the setting section 11A randomly sets a mode of alpha blending in which transparentizing is carried out. Specifically, the setting section 11A may set, by randomly selecting, one of a plurality of modes (e.g., multiplication, addition, and the like) which have been determined in advance. For example, the setting section 11A randomly sets a parameter which defines intensity of the set mode. Specifically, the setting section 11A sets, for each pixel of a margin in a foreground image and for each pixel of a margin in a background image, transmittancy used in the mode of alpha blending.

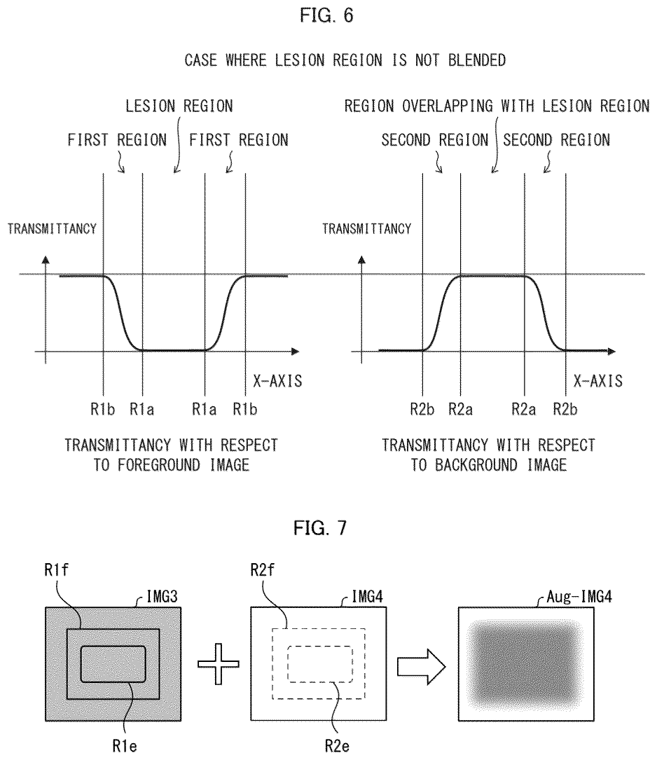

The following description will discuss a specific example of transmittancy to be set for each pixel, with reference to FIG. 6. FIG. 6 is a schematic diagram illustrating a setting example of transmittancy. In FIG. 6, a horizontal axis indicates an x-axis in a foreground image and a background image, and a vertical axis indicates transmittancy. The setting section 11A sets, in the foreground image, a minimum value (i.e., opaque) as transmittancy for each pixel in a lesion region (an inner side of the boundary R1a). The setting section 11A sets, as transmittancy, a maximum value (i.e., transparent) for each pixel in a region (outside the boundary R1b) outside the margin (a first region illustrated in FIG. 6) in the foreground image. The setting section 11A sets, for each pixel of the margin in the foreground image, transmittancy so that the transmittancy becomes closer to a minimum value as a pixel approaches the inner boundary R1a, and the transmittancy becomes closer to a maximum value as a pixel approaches the outer boundary R1b, that is, the transmittancy changes from opaque to transparent from the inside to the outside.

Moreover, the setting section 11A sets, in the background image, a maximum value (i.e., transparent) as transmittancy for each pixel in a region (an inner side of the boundary R2a) which overlaps with a lesion. The setting section 11A sets, as transmittancy, a minimum value (i.e., opaque) for each pixel in a region (outside the boundary R2b) outside the margin (a second region illustrated in FIG. 6) in the background image. The setting section 11A sets, for each pixel of the margin in the background image, transmittancy so that the transmittancy becomes closer to a maximum value as a pixel approaches the inner boundary R2a, and the transmittancy becomes closer to a minimum value as a pixel approaches the outer boundary R2b, that is, the transmittancy changes from transparent to opaque from the inside to the outside.

In step S107, the processing section 12A applies image processing to the foreground image using the processing parameter which has been randomly set in step S106. For example, the processing section 12A applies smoothing to the margin in the foreground image using a type of smoothing filter and a kernel size which have been set. The processing section 12A calculates, based on the transmittancy set in the foreground image, a pixel value for alpha blending with the background image.

In step S108, the processing section 12A applies image processing to the background image using the processing parameter which has been randomly set in step S106. A specific example of the image processing to be applied is similar to step S107, and therefore detailed descriptions thereof will not be repeated. Note that the processes of steps S107 and S108 may be carried out in parallel or may be carried out in the reverse order.

In step S109, the processing section 12A applies image processing to the foreground image and the background image for matching image quality of the foreground image to that of the background image. Examples of image processing for matching image qualities include, but not limited to, a color tone correction process for matching color tones, a blurring process for matching blurring degrees, and the like. For example, the processing section 12A refers to a color tone of the foreground image and a color tone of the background image, and applies, to the foreground image and the background image, a color tone correction process for matching these color tones. For example, the processing section 12A refers to a blurring degree of the foreground image and a blurring degree of the background image, and applies, to the foreground image and the background image, a blurring process for matching the blurring degrees.

In step S110, the generation section 13A generates an augmented medical image by carrying out a process of pasting the processed foreground image to the processed background image. The process of pasting may be, for example, a process of adding pixel values of the processed foreground image and pixel values of the processed background image together. The following description will discuss an augmented medical image, with reference to FIG. 5. For example, an augmented medical image Aug-IMG1 is generated by pasting the image IMG1 which has been subjected to image processing using the margin defined by the boundary R1b to the image IMG2 which has been subjected to image processing using the margin defined by the boundary R2b. For example, an augmented medical image Aug-IMG2 is generated by pasting the image IMG1 which has been subjected to image processing using the margin defined by the boundary Ric to the image IMG2 which has been subjected to image processing using the margin defined by the boundary R2c. For example, an augmented medical image Aug-IMG3 is generated by pasting the image IMG1 which has been subjected to image processing using the margin defined by the boundary R1d to the image IMG2 which has been subjected to image processing using the margin defined by the boundary R2d. As indicated in this example, the augmented medical images Aug-IMG1 through Aug-IMG3 which are different from each other are generated from the same image IMG1 and the same image IMG2 using randomly set processing parameters.

In step S111, the generation section 13A decides a label to be associated with each pixel of an augmented medical image by referring to a label associated with the foreground image, a label associated with the background image, and a processing parameter.

The following description will discuss a specific example of a process of deciding a label. Hereinafter, a region on an augmented medical image to which a lesion region in a foreground image is pasted is referred to as a lesion region in an augmented medical image. Moreover, a region on an augmented medical image to which a margin in a foreground image is pasted is referred to as a margin in an augmented medical image. Moreover, a region on an augmented medical image corresponding to an outer side of a margin in a background image is referred to as an outer side of a margin in an augmented medical image. For example, the generation section 13A decides a label which has been associated with each pixel of a lesion region in a foreground image as a label to be associated with each pixel of a lesion region in an augmented medical image. The generation section 13A decides a label which has been associated with each pixel outside a margin in a background image as a label to be associated with each pixel outside a margin on an augmented medical image. The generation section 13A decides, as a label to be associated with each pixel of a margin in an augmented medical image, a label according to transmittancy set for a margin in a foreground image. Specifically, the generation section 13A calculates, for each pixel, a value of 0 or more and 1 or less as a label such that smaller transmittancy (closer to opacity) is closer to “1” indicating a lesion, and greater transmittancy (closer to transparency) is closer to “0” indicating a non-lesion part.

Thus, each pixel of the lesion region in the augmented medical image is associated with a label indicating a lesion. Moreover, each pixel of the margin in the augmented medical image is associated with a label that approaches from 1 to 0 from the inside to the outside. Each pixel outside the margin in the augmented medical image is associated with a label which has been associated with the original background image.

The generation section 13A causes the augmented image storage apparatus 4 to store the augmented medical image with which labels have been associated. The augmented medical image includes a lesion region and serves as positive training data for training the image recognition model ML.

The image generation apparatus 1A repeats the processes of steps S101 through S111 until a predetermined termination condition is satisfied. Thus, a plurality of augmented medical images are generated and stored in the augmented image storage apparatus 4. The predetermined termination condition may be, but not limited to, that the number of generated augmented medical images exceeds a threshold value, a processing time exceeds a threshold value, and the like.

The training section 21 in the training apparatus 2 trains the image recognition model ML using medical images stored in the image storage apparatus 3 and augmented medical images stored in the augmented image storage apparatus 4.

### <Example Advantage of Present Example Embodiment>

The present example embodiment employs, in addition to a configuration similar to the first example embodiment, the configuration in which a label to be associated with an augmented medical image is decided by referring to a label associated with a foreground image. Thus, in a case where the foreground image includes a lesion region, a generated augmented medical image can be used as positive training data of the lesion region.

Moreover, the present example embodiment employs the configuration of processing one or both of a foreground image and a background image by applying image processing to one or both of a margin including a boundary of a lesion region in the foreground image and a margin including a boundary of a region which, in the background image, overlaps with the lesion region in a case where the foreground image is pasted. Thus, it is possible to set a margin which is a region for reducing influence of a processing process remaining in an augmented medical image.

Moreover, the present example embodiment employs the configuration of deciding a label to be associated with an augmented medical image by further referring to a label associated with a background image and a randomly set processing parameter, in addition to a label associated with a foreground image. Thus, for example, a region of a margin in an augmented medical image can be associated with a label that varies from a label which is associated with an inside region and indicates a lesion to a label which is associated with an outside region and indicates a non-lesion part. As a result, it is possible to reduce a case where a feature of a margin is learned as a feature of a lesion.

The present example embodiment employs the configuration in which the processing parameter includes at least one selected from the group consisting of (1) a parameter which designates a shape of a margin, (2) a parameter which defines a size of one or both of margins, and (3) a parameter which defines image processing. Thus, in a case where a plurality of pieces of training data are generated, margins in the plurality of pieces of training data are uniformly distributed in terms of shape and size without having any tendency. In regard to a trace of image processing which has been applied to margins in the plurality of pieces of training data, features thereof are uniformly distributed without having any tendency. As a result, it is possible to reduce a case where a feature of a margin is learned as a feature of a lesion.

The present example embodiment employs the configuration in which the image processing applied to a margin is smoothing or transparentizing. Thus, it is possible to smooth and transparentize the margin, and it is possible to reduce a case where a feature of the margin is learned as a feature of a lesion.

The present example embodiment employs the configuration of deciding, by referring to a background image, a position at which a foreground image is pasted to the background image. Thus, it is possible to paste a foreground image at an appropriate position corresponding to a background image.

The present example embodiment employs the configuration in which: the foreground image and the background image are each a medical image obtained by imaging a predetermined part of a human body; and, by further referring to constraint information indicating a constraint which pertains to a pasting position corresponding to the predetermined part, a position at which the foreground image is pasted to the background image is decided. Thus, a foreground image which is a medical image can be pasted at an appropriate position of a background image which is a medical image.

### Variation 1

In the above described second example embodiment, the image generation apparatus 1A can be modified to generate, in addition to training data obtained by pasting a lesion region, training data obtained by pasting a dummy region which is not a lesion. In this variation, a margin in a foreground image is a region including a boundary of a dummy region which is not associated with a label indicating a lesion. Note that, as a margin in a background image, a region that is the same as a region which overlaps with the margin in the foreground image is employed, as with the second example embodiment. In this variation, the following steps in the image generation method S1A are modified.

In step S101, the control section 110 selects, as a foreground image, any one of medical images stored in the image storage apparatus 3. The foreground image may or may not include a lesion region.

In step S105, the setting section 11A operates in a manner substantially identical with that in step S105 described above, except for the following points. The setting section 11A randomly decides a dummy region which is associated with a label indicating a non-lesion part in the foreground image. The dummy region only needs to be associated with a label indicating a non-lesion part, and a position, a shape, and a size may be randomly decided.

In step S111, the generation section 13A operates in a manner substantially identical with that in step S111 described above, except for the following points. The generation section 13A decides “0”, which indicates a non-lesion part, as a label to be associated with each pixel of the dummy region and the margin in the augmented medical image. Moreover, the generation section 13A decides a label which has been associated with each pixel outside the margin in the background image as a label to be associated with each pixel outside the margin in the augmented medical image.

Except for the above points, the image generation method S1A in accordance with this variation can be similarly described by replacing a “lesion region” with a “dummy region” in the descriptions of the image generation method S1A in accordance with the second example embodiment described above. Thus, in this variation, in step S111, an augmented medical image which does not include a lesion region is generated as negative training data.

The following description will discuss an augmented medical image generated in this variation, with reference to FIG. 7. FIG. 7 is a schematic diagram illustrating an example of an augmented medical image to which a dummy region is pasted. In the example illustrated in FIG. 7, an image IMG3 which does not include a lesion region is selected as a foreground image. In the image IMG3, a dummy region surrounded by a boundary R1e is set. In addition, in the image IMG3, a margin is set which includes the boundary R1e as an inner boundary and a boundary R1f as an outer boundary. Moreover, as a background image, an image IMG4 which does not include a lesion region is selected. In addition, in the image IMG4, a margin is set which includes a boundary R2e as an inner boundary and a boundary R2f as an outer boundary. The boundary R1e and the boundary R2e overlap with each other in a case where the foreground image is pasted to the background image at a pasting position. Moreover, the boundary R2e and the boundary R2f overlap with each other in a case where the foreground image is pasted to the background image at the pasting position. The image IMG3 is subjected to image processing in accordance with a processing parameter, and the image IMG4 is subjected to image processing in accordance with a processing parameter. After that, the image IMG3 is pasted to the image IMG4, and thus an augmented medical image Aug-IMG4 is generated.

Each pixel of the augmented medical image Aug-IMG4 is associated with a label 0 indicating a non-lesion part. Such an augmented medical image Aug-IMG4 can include a feature of discontinuity in a margin, and it is possible to use the augmented medical image Aug-IMG4 as negative training data indicating a non-lesion part. By using this variation in addition to the second example embodiment, negative training data is stored in the augmented image storage apparatus 4 in addition to positive training data.

The training section 21 in the training apparatus 2 is configured similarly to the second example embodiment. In addition, the training section 21 trains the image recognition model ML using an augmented medical image to which a dummy region is pasted. Both the positive training data and the negative training data may include a feature of discontinuity near a boundary of a lesion region (or dummy region). Therefore, it is possible to reduce a case where a feature of discontinuity is learned as a feature of a lesion, and thus it is possible to improve accuracy of the image recognition model ML.

This variation employs the configuration of processing one or both of a foreground image and a background image by applying image processing to one or both of a margin including a boundary of a dummy region which is not associated with a label indicating a lesion in the foreground image and a margin including a boundary of a region which, in the background image, overlaps with the dummy region in a case where the foreground image is pasted. Thus, it is possible to generate, as negative training data, an augmented medical image which includes a margin with discontinuity and which does not include a lesion region inside the margin. As a result, it is possible to reduce a case where a feature of discontinuity is learned as a feature indicating a lesion.

### Variation 2

In the above described second example embodiment, the processing section 12A may apply image processing to a region inside a margin. The region inside the margin is a lesion region in a foreground image and is a region which overlaps with the lesion region in a background image. In this variation, the following steps in the image generation method S1A are modified.

In step S105, the setting section 11A operates in a manner substantially identical with that in step S105 described above, except for the following points. The setting section 11A randomly sets, in a foreground image, a processing parameter for a lesion region in addition to a processing parameter for a margin. Moreover, the setting section 11A randomly sets, in a background image, a processing parameter for a region which overlaps with the margin lesion region, in addition to a processing parameter for a margin.

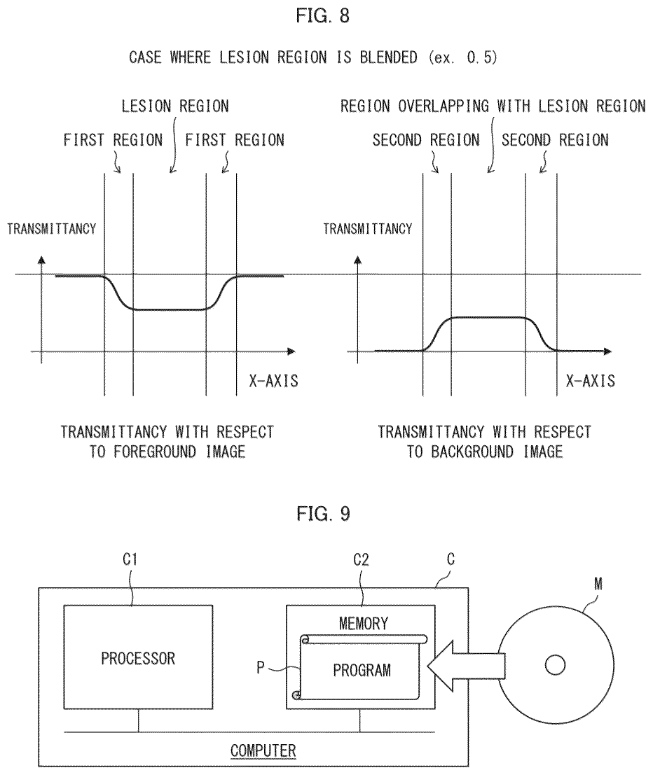

The following description will discuss a specific example of transmittancy which is set for each pixel of the lesion region and the region which overlaps with the lesion region in this variation, with reference to FIG. 8. FIG. 8 is a schematic diagram illustrating a setting example of transmittancy. In FIG. 8, a horizontal axis indicates an x-axis in a foreground image and a background image, and a vertical axis indicates transmittancy. The setting section 11A sets, in the foreground image, translucent transmittancy (e.g., 50%) for each pixel in the lesion region (inside the boundary R1a). In the foreground image, for each pixel in a region (outside the boundary R1b) outside the margin, a maximum value (i.e., transparent) is set as transmittancy. The setting section 11A sets, for each pixel of the margin in the foreground image, transmittancy SO that the transmittancy becomes closer to 50% as a pixel approaches the inner boundary R1a, and the transmittancy becomes closer to a maximum value as a pixel approaches the outer boundary R1b, that is, the transmittancy changes from translucent to transparent from the inside to the outside.

The setting section 11A sets, in the background image, translucent transmittancy (e.g., 50%) for each pixel in a region (inside the boundary R2a) which overlaps with the lesion region. The setting section 11A sets, as transmittancy, a minimum value (i.e., opaque) for each pixel in a region (outside the boundary R2b) outside the margin in the background image. The setting section 11A sets, for each pixel of the margin in the background image, transmittancy so that the transmittancy becomes closer to 50% as a pixel approaches the inner boundary R2a, and the transmittancy becomes closer to a minimum value as a pixel approaches the outer boundary R2b, that is, the transmittancy changes from translucent to opaque from the inside to the outside.

In step S111, the generation section 13A operates in a manner substantially identical with that in step S111 described above, except for the following points. The generation section 13A sets, as a label to be associated with each pixel in a lesion region in an augmented medical image, a value obtained by multiplying a label indicating a lesion by transmittancy. For example, in a case where transmittancy set for each pixel in a lesion region is 50%, a label is 0.5. The generation section 13A decides a label which has been associated with each pixel outside a margin in a background image as a label to be associated with each pixel outside a margin in an augmented medical image. The generation section 13A decides, as a label to be associated with each pixel of the margin in the augmented medical image, a label according to transmittancy set for a margin in a foreground image.

Thus, for example, a label “0.5” is associated with each pixel of the lesion region in the augmented medical image. Moreover, each pixel of the margin in the augmented medical image is associated with a label that approaches from 0.5 to 0 from the inside to the outside. Each pixel outside the margin in the augmented medical image is associated with a label which has been associated with the original

According to this variation, the configuration is employed in which image processing is applied to a lesion region in the foreground image and a region which overlaps with the lesion region in the background image, in addition to margins in the foreground image and the background image. Thus, in the generated augmented medical image, it is possible to further reduce a difference in feature between the lesion region and the region outside thereof, and thus further to alleviate discontinuity.

Note that this variation can also be combined with Variation 1. That is, the processing section 12A may apply image processing to a dummy region in a foreground image and a region which overlaps with the dummy region in a background image, in addition to margins in the foreground image and the background image.

### Other Variation

In the above described second example embodiment, an example has been described in which smoothing or transparentizing is carried out as image processing. The second example embodiment is not limited to the example, and the processing section 12A may apply other image processing to one or both of a foreground image and a background image in place of or in addition to smoothing or transparentizing.

In the foregoing example embodiments, in a case where a plurality of types of image processing are applied to one or both of the foreground image and the background image, a margin may be set in accordance with the type of image processing. For example, a margin to which transparentizing is applied and a margin to which a smoothing filter is applied may differ in terms of at least one of a shape, a size, and a position.

In the above described second example embodiment, it has been described that a margin in the background image is the same as a region which overlaps with a margin in the foreground image. Note, however, that the margins may not necessarily be the same. For example, the margin in the background image may be a region which includes the margin in the foreground image and is wider than the margin in the foreground image.

In the above described second example embodiment, it has been described that the margin in the foreground image is a region which includes a boundary of a lesion region as an inner boundary, i.e., is a region outside the lesion region. The second example embodiment is not limited to the example, and the margin in the foreground image may include a region inside the lesion region. The same applies to the margin in the background image.

In the above described second example embodiment, an example has been described in which image processing applied to a foreground image is the same as that applied to a background image. The second example embodiment is not limited to the example, and at least a part of the image processing applied to the foreground image may be different from the image processing applied to the background image. For example, smoothing and transparentizing may be applied to the foreground image, and transparentizing may be applied to the background image without smoothing.

In the above described second example embodiment, an example has been described in which types identified by labels are two types, i.e., being a lesion or not. The second example embodiment is not limited to the example, and types to be identified by labels may be three or more types (e.g., a first type lesion, a second type lesion, a non-lesion, and the like). In this case, the generation section 13A may decide a label to be associated with each pixel of a margin in an augmented medical image as follows. For example, it is assumed that a first label is associated with a region inside a margin in a foreground image, and a second label is associated with a region outside a margin in a background image. For example, the generation section 13A may associate, with each pixel of the margin in the augmented medical image, a weight for the first label and a weight for the second label, instead of associating a single label. Such a weight is set in accordance with transmittancy set for each pixel of the margin in the foreground image.

The above described second example embodiment has been described as generating training data for use in machine learning of an image recognition model which carries out a segmentation task. The second example embodiment is not limited to the example, and is also applicable to a case where training data is generated for use in machine learning which carries out a detection task of detecting a rectangle region including a lesion, a classification task of recognizing a class of a lesion for each image, and the like.

In the case of the classification task, a label is associated with each medical image. Therefore, the generation section 13A decides, as a label to be associated with the generated augmented medical image, a label which has been associated with the foreground image. In the case of the detection task, a medical image which is training data is associated with information of a rectangle region including a lesion and a label corresponding to each rectangle region. Therefore, the generation section 13A decides a label associated with a rectangle region including a lesion region in a foreground image as a label to be associated with a rectangle region including a lesion region in an augmented medical image. The generation section 13A associates, with a region outside a margin in the augmented medical image, a rectangle region which has been associated with a region outside a margin in a background image and a label thereof.

The above described second example embodiment is also applicable to a case where an image other than a medical image is generated as training data. For example, the present example embodiment can generate training data for recognizing a region of interest in an image in which a difference in feature between a region to be recognized and a region of non-interest is small, in other words, an image in which it is difficult to distinguish between the region to be recognized and the region of non-interest. In such an image, it is particularly important to reduce a case where a feature of discontinuity at a boundary between a region of interest in a foreground image and a background image is learned as a feature of the subject. Examples of such an image and a subject to be recognized include an example in which an object (a car, a person, a forest, a farm land, a road, or the like) is recognized in an aerial photograph. The aerial photograph may be captured by a visible light camera or may be captured by a radar mounted on an artificial satellite, an aircraft, or the like. An image captured by a radar is generated by transforming reflection of the radar into a two-dimensional image. Therefore, there is a tendency that it is further difficult to distinguish between an object and surroundings, as compared with an image captured by a visible light camera. Note, however, that types of images other than a medical image which are applicable in the present example embodiment are not limited to the above described examples.

### Software Implementation Example

Some or all of the functions of the image generation apparatuses 1 and 1A and the training apparatus 2 (hereinafter each of the apparatuses are referred to as the “above apparatus”) may be implemented by hardware such as an integrated circuit (IC chip), or may be implemented by software.

In the latter case, the above apparatus is implemented by, for example, a computer that executes instructions of a program that is software implementing the functions of the above apparatus. FIG. 9 illustrates an example of such a computer (hereinafter, referred to as “computer C”). The computer C includes at least one processor C1 and at least one memory C2. The memory C2 stores a program P for causing the computer C to function as the above apparatus. The processor C1 of the computer C retrieves the program P from the memory C2 and executes the program P, so that the functions of the above apparatus are implemented.

As the processor C1, for example, it is possible to use a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these. Examples of the memory C2 include a flash memory, a hard disk drive (HDD), a solid state drive (SSD), and a combination thereof.

Note that the computer C can further include a random access memory (RAM) in which the program P is loaded in a case where the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a computer C-readable, non-transitory, and tangible storage medium M. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communication network, a broadcast wave, or the like. The computer C can obtain the program P also via such a transmission medium.

### Additional Remark 1

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### Additional Remark 2

Some or all of the foregoing example embodiments can also be described as below. Note, however, that the present invention is not limited to the following supplementary notes.

### Supplementary Note 1

An image generation apparatus, including: a setting means for randomly setting a processing parameter; a processing means for processing one or both of a first image and a second image in accordance with the processing parameter; and a generation means for pasting the first image to the second image so as to generate a third image that serves as training data for machine learning.

### Supplementary Note 2

The image generation apparatus according to supplementary note 1, in which: the generation means decides a label to be associated with the third image by referring to a label associated with the first image.

### Supplementary Note 3

The image generation apparatus according to supplementary note 2, in which: the generation means decides a label to be associated with the third image by referring to a label associated with the second image and the processing parameter, in addition to the label associated with the first image.

### Supplementary Note 4

The image generation apparatus according to any one of supplementary notes 1 through 3, in which: the processing means processes one or both of the first image and the second image by applying image processing to one or both of a first region and a second region, the first region including a boundary of a predetermined region which is associated with a predetermined label in the first image, and the second region including a boundary of a region which, in the second image, overlaps with the predetermined region in a case where the first image is pasted.

### Supplementary Note 5

The image generation apparatus according to any one of supplementary notes 1 through 3, in which: the processing means processes one or both of the first image and the second image by applying image processing to one or both of a first region and a second region, the first region including a boundary of a dummy region which is not associated with a predetermined label in the first image, and the second region including a boundary of a region which, in the second image, overlaps with the dummy region in a case where the first image is pasted.

### Supplementary Note 6

The image generation apparatus according to supplementary note 4 or 5, in which: the processing parameter includes at least one selected from the group consisting of (1) a parameter which designates a shape of one or both of the first region and the second region, (2) a parameter which defines a size of one or both of the first region and the second region, and (3) a parameter which defines the image processing.

### Supplementary Note 7

The image generation apparatus according to any one of supplementary notes 4 through 6, in which: the image processing is smoothing or transparentizing.

### Supplementary Note 8

The image generation apparatus according to any one of supplementary notes 1 through 7, further including: a decision means for deciding, by referring to the second image, a position at which the first image is pasted to the second image.

### Supplementary Note 9

The image generation apparatus according to supplementary note 8, in which: the first image and the second image are each a medical image obtained by imaging a predetermined part of a human body; and the decision means decides, by further referring to constraint information indicating a constraint which pertains to a pasting position corresponding to the predetermined part, a position at which the first image is pasted to the second image.

### Supplementary Note 10

A training apparatus, including: a training means for training an image recognition model using training data which has been generated by using the image generation apparatus according to any one of supplementary notes 1 through 9.

### Supplementary Note 11

An image generation method, including: randomly setting, by at least one processor, a processing parameter; processing, by the at least one processor, one or both of a first image and a second image in accordance with the processing parameter; and pasting, by the at least one processor, the first image to the second image so as to generate a third image that serves as training data for machine learning.

### Supplementary Note 12

A program for causing at least one processor to function as: a setting means for randomly setting a processing parameter; a processing means for processing one or both of a first image and a second image in accordance with the processing parameter; and a generation means for pasting the first image to the second image so as to generate a third image that serves as training data for machine learning.

### Additional Remark 3

Furthermore, some of or all of the foregoing example embodiments can also be expressed as below.

### Supplementary Note 13

An image generation apparatus, including at least one processor, the at least one processor carrying out: a setting process of randomly setting a processing parameter; a processing process of processing one or both of a first image and a second image in accordance with the processing parameter; and a generation process of pasting the first image to the second image so as to generate a third image that serves as training data for machine learning.

Note that the image generation apparatus may further include a memory. The memory may store a program for causing the at least one processor to carry out the setting process, the processing process, and the generation process. The program may be stored in a computer-readable non-transitory tangible storage medium.

### Supplementary Note 14

A training apparatus, including at least one processor, the at least one processor carrying out: a training process of training an image recognition model using training data which has been generated by using the image generation apparatus according to supplementary note 13.

Note that the training apparatus may further include a memory. The memory may store a program for causing the at least one processor to carry out the training process. The program may be stored in a computer-readable non-transitory tangible storage medium.

### REFERENCE SIGNS LIST

100: Information processing system
1, 1A: Image generation apparatus
2: Training apparatus
3: Image storage apparatus
4: Augmented image storage apparatus
11, 11A: Setting section
12, 12A: Processing section
13, 13A: Generation section
14, 14A: Decision section
15A: Preprocessing section
21: Training section
110, 210: Control section
120, 220: Storage section
C1: Processor
C2: Memory

What is claimed is:

1. An image generation apparatus, comprising at least one processor, the at least one processor carrying out:
a setting process of randomly setting a processing parameter;
a processing process of processing one or both of a first image and a second image in accordance with the processing parameter; and
a generation process of pasting the first image to the second image so as to generate a third image that serves as training data for machine learning.

2. The image generation apparatus according to claim 1, wherein:
in the generation process, the at least one processor decides a label to be associated with the third image by referring to a label associated with the first image.

3. The image generation apparatus according to claim 2, wherein:
in the generation process, the at least one processor decides a label to be associated with the third image by referring to a label associated with the second image and the processing parameter, in addition to the label associated with the first image.

4. The image generation apparatus according to claim 1, wherein:
in the processing process, the at least one processor processes one or both of the first image and the second image by applying image processing to one or both of a first region and a second region, the first region including a boundary of a predetermined region which is associated with a predetermined label in the first image, and the second region including a boundary of a region which, in the second image, overlaps with the predetermined region in a case where the first image is pasted.

5. The image generation apparatus according to claim 1, wherein:
in the processing process, the at least one processor processes one or both of the first image and the second image by applying image processing to one or both of a first region and a second region, the first region including a boundary of a dummy region which is not associated with a predetermined label in the first image, and the second region including a boundary of a region which, in the second image, overlaps with the dummy region in a case where the first image is pasted.

6. The image generation apparatus according to claim 4, wherein:
the processing parameter includes at least one selected from the group consisting of (1) a parameter which designates a shape of one or both of the first region and the second region, (2) a parameter which defines a size of one or both of the first region and the second region, and (3) a parameter which defines the image processing.

7. The image generation apparatus according to claim 4, wherein:
the image processing is smoothing or transparentizing.

8. The image generation apparatus according to claim 1, wherein:
the at least one processor further carries out a decision process of deciding, by referring to the second image, a position at which the first image is pasted to the second image.

9. The image generation apparatus according to claim 8, wherein:
the first image and the second image are each a medical image obtained by imaging a predetermined part of a human body; and
in the decision process, the at least one processor decides, by further referring to constraint information indicating a constraint which pertains to a pasting position corresponding to the predetermined part, a position at which the first image is pasted to the second image.

10. A training apparatus, comprising at least one processor, the at least one processor carrying out:
a training process of training an image recognition model using training data which has been generated by using an image generation apparatus according to claim 1.

11. An image generation method, comprising:
randomly setting, by at least one processor, a processing parameter;
processing, by the at least one processor, one or both of a first image and a second image in accordance with the processing parameter; and
pasting, by the at least one processor, the first image to the second image so as to generate a third image that serves as training data for machine learning.

12. A non-transitory storage medium storing a program for causing at least one processor to carry out:
a setting process of randomly setting a processing parameter;
a processing process of processing one or both of a first image and a second image in accordance with the processing parameter; and
a generation process of pasting the first image to the second image so as to generate a third image that serves as training data for machine learning.
